(19) United States
(12) Patent Application Publication
(10) Pub. No.: US 2022/0152144 A1
(43) Pub. Date: May 19, 2022

(54) FOOD ADDITIVE INCLUDING MANNAN OLIGOSACCHARIDES TO ENHANCE ANIMAL PERFORMANCE AND METHOD OF USING THE SAME

(71) Applicant: Vitech Bio-Chem Corporation, Orange, CA (US)

(72) Inventor: Thomas Shau Shieh, Orange, CA (US)

(21) Appl. No.: 17/526,861

(22) Filed: Nov. 15, 2021

Related U.S. Application Data
(60) Provisional application No. 63/113,290, filed on Nov. 13, 2020.

(51) Int. Cl. A61K 38/01; A61K 31/702; A61P 3/02; A23K 20/147; A23K 20/163; A23K 50/75

(52) U.S. Cl. CPC A61K 38/012; A61K 31/702; A23K 50/75; A23K 20/147; A23K 20/163; A61P 3/02

(57) ABSTRACT

Provided here is a feed supplement composition for animal food products comprising a fish-derived hydrolysate peptide (FHP) and a mannan oligosaccharide (MOS) are provided herein. The combination was found to result in unexpected increases in feed intake, feed conversion and body weight gain when used in an animal feed. Also provided are methods of using an animal feed comprising FHP and MOS to improve poultry agriculture performance.

### CROSS-REFERENCE TO RELATED APPLICATIONS

This application claims priority to U.S. Provisional Application No. 63/113,290 filed on Nov. 13, 2020, the contents of which are incorporated by reference in its entirety.

### INTRODUCTION

Antibiotics have traditionally been used in the animal protein farming industry to reduce the impact of bacterial infections and to improve animal growth performance. Due to increasing concerns about the rise of antibiotic resistance in human health, many countries have banned the prophylactic use of antibiotic growth promoters (AGP) in animal feed (Gadde, U., et al., Anim Health Res Rev. 18(1): 26-45 (2017)). This has resulted in a decrease in animal performance and a rise in the incidence of illnesses associated with bacterial infections such as Clostridium perfringens, Salmonella, Escherichia coli, etc. As a result, the industry is a looking for alternatives to antibiotics (Gadde et al., 2017).

The present disclosure aims to solve these concerns and proposes an alternative to antibiotics with a feed supplement comprising a fish-derived hydrolysate peptide (FHP) and mannan oligosaccharides (MOS).

### SUMMARY

Provided herein are feed supplements and animal food products that satisfies the need of providing to animals products that do not contain antibiotics.

In one aspect, provided herein is a feed supplement comprising a fish-derived hydrolysate peptide (FHP) composition and mannan oligosaccharides (MOS). The fish-derived hydrolysate peptide composition may comprise 40-80% of protein, 4-10% of fat, and/or 0.5-10% of fiber.

In another aspect, provided herein is an animal food product comprising a fish-derived hydrolysate peptide (FHP) composition and mannan oligosaccharides (MOS). The animal food product may comprise a feed supplement described herein. The animal food product may comprise 0.01-10% of a feed supplement described herein.

In another aspect, provided herein are methods for feeding domestic animals by supplying the feed supplement or the animal food product to domestic animals are provided herein. These methods include domestic animals which are poultry.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

These and other features, aspects, and advantages of the present invention will become better understood with regard to the following description, appended claims, and accompanying drawings.

FIG. 1 is a graph showing weight gain (g) of finisher phase broiler chicks fed a traditional diet supplemented with avilamycin (Control group) or 3 different versions of the feed supplements described herein having different doses of MOS in parts per million (ppm).

FIG. 2 is a graph showing mortality rates (%) of broiler chickens when subjected to diets having avilamycin and various versions of the feed supplements described herein. NC stands for negative control, in which no supplementation was provided to the chicken diet.

FIG. 3 is a graph showing feed conversion ratios ((FCR): ratio of kg of feed to produce 1 kg of body weight/egg weight) for diets containing various versions of the feed supplements described herein. The FHP+MOS diets showed better FCRs compared to traditional diets supplemented with enramycin (Enramycin) or no additives (NC). As the kg of feed weight to produce 1 kg of egg (FCR) decreases, the economic benefit increases.

### DETAILED DESCRIPTION

Antibiotics have traditionally been used in animal protein farming industry to reduce the impact of bacterial infections and for improving animal growth performance. Due to increasing concerns about rise of antibiotic resistance in human health, many countries have banned the prophylactic use of antibiotic growth promoters (AGP) in animal feed (Gadde et al., 2017). Therefore, the industry is looking for alternatives to antibiotics (Gadde et al., 2017). Provided herein are feed supplements and animal food products that address the shortcomings in the industry.

Porcine-derived hydrolysate peptides have been suggested to increase voluntary feed consumption in nursery pigs, and piglets were comparable to those fed diets that contained whey (Solà-Oriol, D., et al., J Animal Sci. 89: 3219 (2011)) but were less preferred when compared to those pigs fed lactose (Figueroa, J., et al., J Anim Sci. 94: 1531 (2016)). Fish-derived hydrolysate peptides (FHP) or known as fish hydrolysate (FH), such as Peptiva® (Vitech Bio-Chem, Corp, CA), were reported to improve intake. These data are in agreement with findings of Norgaard et al., (Animal Feed Science and Technology 177(1-2): 124-9 (2012)) and effectively restored weight gain when compared to those pigs fed with spray dried plasma protein (SDPP) in trials that were conducted in Virginia and Georgia. This was especially true when the amino acids were balanced to an ideal ratio. In addition, economic return was higher in Peptiva® than SDPP diets. In field trials, it was suggested that a combination of Peptiva® and probiotics/prebiotic could potentially have synergistic effects.

The present inventor has surprisingly found that fish-derived hydrolysate peptides (FHPs) in combination with mannan oligosaccharides (MOS) can be used to replace antibiotics. A combination of FHPs and MOS can also have a synergistic effect on performance, improve blood cell count and fend off bacteria. Thus, provided herein is a feed supplement that contains a FHP and MOS.

FHPs are commercially available. FHP suppliers include, but are not limited to, Vitech Bio-Chem Corporation (California, U.S.A.), Apelsa (Guadalajara, Mexico), Qingdao Future Group (Shangond, China), Mukka Seafood Industries Limited (India), Ocean Protein (Washington, USA), Omega Protein Corporation (Texas, U.S.A.), Daybrook Fisheries, Inc. (Louisiana, USA), and Blue Wave Marine (Lima, Peru).

The fish-derived hydrolysate peptide composition used in the feed supplement disclosed herein may contain from 40 to 80% of protein. The fish-derived hydrolysate peptide composition may also contain from 4 to 10% of fat and from 0.5 to 10% of fiber.

Mannan oligosaccharides (MOS) are derived from yeast cell walls and are used as probiotics/prebiotics by the animal feed industry to improve farm animal health and performance. MOS are increasingly being applied as antibiotic replacers for growth promotion. Yeast cell walls are rich in two natural functional polysaccharides with well accepted health improving properties: MOS and (1,3)(1,6)-β-D-glucan. MOS, present on the outer layer of autolyzed yeast cell walls (van der Werf, MOS Products: Not Every Yeast Cell Wall Is Created Equal, www.ohly.com, 2019), bind pathogenic bacteria thereby inhibiting their colonization of the gut and thus preventing infections or the release of toxins (Kwiatkowski and Kwiatkowski, Yeast (Saccharomyces cerevisiae) Glucan Polysaccharides, (2012); Fowler, J., et al., Toxins 7: 3455 (2015)). It has also been shown that β-glucans improve the resistance of farm animals against microbial infections.

Separately in pigs, weaning stress includes changes in diet form and social interaction, such as competing dominance order and isolation from dam, which often results in reduction in intake and breaks in intestinal barrier integrity. This allows opportunistically pathogenic bacteria to become dominant in the commensal microflora community and leads to post weaning diarrhea, growth retardation, and even death. Therefore, early piglet nursery diets are typically formulated using ingredients that are not only highly bioavailable but also palatable to serve as means to stimulate intake in order to provide sufficient amount of nutrients to increase recovery rate and attenuate negative impact from weanling process.

In one aspect of the invention, the feed supplement contains MOS. Mannan oligosaccharides (MOS) or yeast cell walls (YCW) comprising MOS are commercially available. MOS can be obtained from Feed Sources (Sherman, Tex.), AllTech, (Nicholasville, Ky.), Lallemand. Inc. (Ontario, Canada), Marsyt Corporation (Elizabethtown, Pa.), and Trouw Nutrition (Strykersville, N.Y.). In one aspect, the feed supplement also contains yeast cell walls available from brewer's yeast suppliers.

The results of studies provided herein regarding feeding animals supplements containing fish hydrolysate (FH) and MOS were positive on using along with or replacing antibiotic growth promoters (AGP). The studies showed that the combination of FH and MOS (FH+MOS) is better than using MOS alone in improving animal growth performances. The results of these studies are shown in the Examples below.

The feed supplement disclosed herein may have a ratio of 1:1 to 1,000:1 by weight of FHP composition to MOS. In one aspect, the feed supplement disclosed herein has a ratio of 1:1 to 100:1 by weight of fish-derived hydrolysate peptide composition to yeast cell walls (MOS). In one aspect, the feed supplement disclosed herein has a ratio of 1:1 to 10:1 by weight of FHP composition to yeast cell wall material comprising MOS. In one aspect, the feed supplement disclosed herein has a ratio of 1:1 to 4:1 by weight of FHP composition to yeast cell wall material comprising MOS.

A feed supplement or animal food product disclosed herein may be supplied to a subject. In some embodiments, a method of the present disclosure comprises supplying to a subject a feed supplement composition or animal food product of the present disclosure. The term “subject” includes human and non-human subjects. In some embodiments, the subject is an animal. In some embodiments, the subject is a mammal, such as a human, dog, cat, or livestock, such as cattle, goats, pigs, or sheep. In some embodiments, the subject is a fish. In some embodiments, the subject is a wild, domesticated, or captive population of animals, such as deer, elk, bison, etc. In some embodiments, the subject may include one or more wild or domesticated birds, such as chickens, ducks, or turkeys. Non-limiting examples of a subject include swine, poultry, domestic pets and aquacultures. Swine includes, but is not limited to, nursery piglets, breeding pigs, gestational sows, lactating sows, growing-finishing pigs, or starter pigs. Poultry includes, but is not limited to, broiler chicks, grower chicks, finisher chicks, layer poults, egg laying hens, turkey poults, young turkey, or grower-finisher turkey. Domestic pets include, but are not limited to, cats and dogs.

In some aspects of the invention, the feed supplement is given to broiler chickens. In other aspects of the invention, the feed supplement is given to egg laying hens. Broiler chicks are usually fed chick starter diet for 14 days, grower diet is fed from 15 days from hatch to 28 days old, finisher diet is fed from 29 days old to 42 days old. The broiler life stages can be varied depending on different genetics and farms. Laying hens (layer) and turkey diets also vary by farms and growers, but would be understood by those skilled in the art.

An animal food product is also disclosed herein. The animal food product may be a poultry food product. The animal food product contains the feed supplement disclosed herein. In one aspect of the invention, the feed supplement comprises from 0.01% to 10% of the animal food product. In another aspect, the feed supplement comprises from 0.1% to 2% of the animal food product. The animal food product also comprises corn, soy, fat, limestone powder, amino acids, and vitamins. In one aspect, the amino acids may be in crystalline form. The inclusion rates (dose) found to be effective is between 50 grams per ton (gm/ton) (0.005%) to 100 kg/ton (10%). The inclusion rate may be 100 gm/ton (0.01%) to 50 kg/ton (5.0%) or 100 gm/ton to 20 kg/ton (0.1% to 2%). The animal feed formula may include other ingredients, such as, e.g., limestone, vitamins and trace minerals, enzyme, etc.

The animal food product can be given to, but is not limited to, swine, poultry, domestic pets and aquacultures as noted above for the feed supplement. The food product composition will depend on the animal being fed as will be understood immediately by those of skill in the art.

In some aspects of the invention, the animal food product is given to poultry. In one aspect, the animal food product is given to broiler chickens. In another aspect, the animal food product is given to egg laying hens. Broiler chicks are usually fed chick starter diet for 14 days, grower diet is fed from 15 days from hatch to 28 days old, finisher diet is fed from 29 days ole to 42 days old. The broiler life stages can be varied depending on different genetics and farms. Laying hens (layer) and turkey diets may also vary by farm and grower.

In some aspects, the animal food product does not contain an antibiotic growth promoter (AGP) or is free or substantially free from commercially produced antibiotics.

Methods for feeding poultry by supplying the feed supplement disclosed herein or the animal food product disclosed herein are also provided herein.

Supplying may mean feeding the animal food product to chicken from hatched to market size, egg laying hens from hatched to 2 years old, turkey from hatched to market size, and chicken breeding stocks, or other poultry. It may also mean incorporating the feed supplement into a complete feed and feeding poultry, layer, or turkey the complete feed. Supplying may also mean giving the feed supplement to poultry, layer, or turkey.

The present inventor found that supplying the feed supplement or the animal food product to poultry, layer or turkey resulted in improvements in feed intake, weight gained, and feed conversion rate, mortality rate over poultry, layer or turkey that was fed with industry standard feed or a poultry, and/or layer or turkey food product that lacked the fish-derived hydrolysate peptide composition and antibiotic growth promoter (AGP).

Experiments showed that when the feed supplement or the animal food product are fed to broiler chicks, the broiler chicks had an increase in average daily gain, an increase in body weight, an increase in average daily feed intake, an increase in the ratio of weight gain to feed, or a combination thereof, as compared to the broiler chicks fed with feed supplement or food product lacking the fish-derived hydrolysate peptide composition and MOS.

Further experiments showed that when the feed supplement or the animal food product are fed to broiler chicks, the broiler chicks had a similar average daily gain, a similar increase in body weight, a similar average daily feed intake, a similar ratio of weight gain to feed, or a combination thereof, as compared to the broiler chicks fed with feed supplement or food product containing an antibiotic growth promoter (AGP). Even further, experiments showed that the when the feed supplement or the food product are fed to broiler chicks, the broiler chicks had a decrease in mortality rate of the broiler chicks as compared to comparable boiler chicks fed with feed supplement or food product lacking the fish-derived hydrolysate peptide composition and MOS and/or a feed supplement or food product containing antibiotic growth promoter (AGP).

The present inventor also found that when egg-laying hens were fed the feed supplement or the animal food product, the egg-laying hens had an increase in feed intake compared to the egg-laying hens fed with feed supplement or food product lacking the fish-derived hydrolysate peptide composition and MOS. Further experiments showed that when egg-laying hens were fed the feed supplement or the animal food product, the egg-laying hens had an increase in feed intake of the egg laying hens as compared to the egg laying hens fed with feed supplement or food product containing antibiotic growth promoter (AGP).

Experiments also showed that when egg-laying hens were fed the feed supplement or the animal food product, the egg-laying hens had similar egg laying rates as compared to the egg laying hens fed with feed supplement or food product containing antibiotic growth promoter (AGP).

Further experiments showed that when egg-laying hens were fed the feed supplement or the animal food product provided herein, the egg-laying hens laid eggs that had an increase in egg weight, a decrease in egg feed conversion ratio, or a combination thereof of as compared to eggs from egg laying hens fed with feed supplement or food product lacking the fish-derived hydrolysate peptide composition and MOS. Further experiments showed that when egg-laying hens were fed the feed supplement or the animal food product, the egg-laying hens laid eggs that had an increase in egg weight, a decrease in egg feed conversion ratio, or a combination thereof of eggs laid by the treated egg laying hens as compared to eggs from egg laying hens fed with feed supplement or food product containing antibiotic growth promoter (AGP).

Further experiments showed that when egg-laying hens were fed the feed supplement or the animal food product, the eggs laid by the egg-laying hens had similar eggshell color values, a similar egg yolk, a similar percentage of shell, yolk, and egg white, or a combination thereof of eggs laid from treated egg laying hens as compared to the egg laying hens fed with feed supplement or food product containing antibiotic growth promoter (AGP).

As used herein, the terms “fish protein hydrolysate,” “fish hydrolysate peptide,” “FHP,” or “FH” refers to a composition comprising a breakdown product(s) of fish protein comprising polypeptides and peptides. A FHP may optionally comprise one or more individual amino acids. A FHP may optionally comprise a minority (compared to polypeptide/peptide) of fat or ash.

As used herein, the terms mannan oligosaccharides or MOS refers to a complex carbohydrate comprising glucomannoprotein derived from the outer cell wall of a fungus, such as a Baker's yeast like S. cerevisiae or a Paecilomyces spp. A MOS composition is a composition comprising a plurality of at least one mannan oligosaccharide. Generally, a MOS is not digestible in the digestive system of an animal but serves as a substrate for gastrointestinal microorganisms.

As used herein, the term comparable as referring to a subject means the subject is of a group of the same species, sub-species, breed, strain, or clone such that individuals of the group exhibit a similar applicable performance feature(s), such as similar weight gain, feed intake, weight gain to feed intake ratio, and/or egg FCR for a given diet.

As used herein, the term antibiotic growth promoter (AGP) refers to any medicine that is biocidal or biotic static and is administered or supplied at a subtherapeutic dosage. Non-limiting examples of AGPs include bacitracin, flavophospholipol, lactam antibiotics (e.g. penicillins), lincosamides and macrolides (e.g. erythromycin and tetracyclines), pleuromutilins, quinoxalines, virginiamycin, and arsenical compounds.

An eggshell color value may be determined using a standardized fan labeled with color values which contains color shades for the shell of eggs allowing for variations within a range of colors. The skilled worker will appreciate how to match a given shell color with a color shade in the fan to ascertain an eggshell color value for a given egg. Typically, more brown shades are considered superior.

An egg yolk color may be determined using a color measurement using a standardized fan labeled with color values which contains color shades for egg yolks. Typically, more red shades are considered superior.

As used herein, the terms include and including have the same meaning as the terms comprise and comprising in that these latter terms are open transitional terms that do not limit claims only to the recited elements succeeding these transitional terms. The term consisting of, while encompassed by the term comprising, should be interpreted as a closed transitional term that limits claims only to the recited elements succeeding this transitional term. The term consisting essentially of, while encompassed by the term comprising, should be interpreted as a partially closed transitional term which permits additional elements succeeding this transitional term, but only if those additional elements do not materially affect the basic and novel characteristics of the claim.

The Examples provided below are meant to be illustrative and not to limit the scope of the invention or the claims. All references and appendices cited herein are hereby incorporated by reference in their entireties.

### EXAMPLES

### Example 1. Peptiva® Used in Broiler Diet

This example evaluates the effects of prebiotics on growth performance and microbial changes in broiler chicks.

Materials and Methods. Broiler chicks: A total of 960 1-day old broiler chicks (Name of breed: Cobb500™) (8 trt×6 rep×20 birds/pen) were randomly allocated to 8 treatment groups. Treatment groups: Treatment 1 was a corn/soybean meal diet (PC: 45 ppm avilamycin); Treatment 2 was a PC+50 g/T Fish hydrolysate+MOS (80:20 ratio); Treatment 3 was a PC+100 g/T Fish hydrolysate+MOS (80:20); and treatment 4 was PC+200 g/T Fish hydrolysate+MOS (80:20).

The trial was conducted for 42 days. Basic diet formulation is presented in Table 1. Body weight gain, feed intake, and FCR was measured at 14, 28, and 42 days. Cecal levels of E. coli, Salmonella, and lactobacillus were evaluated at 21 days.

TABLE 1
Feed formulation for broiler starter, grower,
and finisher diets (as-fed basis)

| | Starter | Grower | Finisher |
|---|---|---|---|
| Item | (0-14 d) | (15-28 d) | (29-42 d) |
| Ingredient (% of diet) | | | |
| Corn | 62.37 | 66.76 | 67.80 |
| Soybean meal (48%) | 30.83 | 26.66 | 25.14 |
| Soybean oil | 1.50 | 2.00 | 3.00 |
| Limestone | 1.22 | 1.16 | 1.07 |
| Defluorinated phosphate | 1.70 | 1.56 | 1.36 |
| Salt | 0.30 | 0.30 | 0.30 |
| Vitamin mix | 0.25 | 0.25 | 0.25 |
| Mineral mix | 0.08 | 0.08 | 0.08 |
| DL-Methionine | 0.33 | 0.28 | 0.23 |
| L-Lysine | 0.33 | 0.31 | 0.18 |
| Threonine | 0.10 | 0.09 | 0.04 |
| Coban 90 | 0.05 | 0.05 | 0.05 |
| Sand | 0.94 | 0.51 | 0.50 |
| ME (kcal/kg) | 3,000 | 3,090 | 3,170 |
| CP (%) | 21.02 | 19.32 | 18.49 |
| Ca (%) | 0.90 | 0.84 | 0.76 |
| Available P (%) | 0.45 | 0.42 | 0.38 |
| Met and Cys (%) | 0.98 | 0.89 | 0.82 |
| Met (%) | 0.64 | 0.57 | 0.52 |
| Lys (%) | 1.32 | 1.19 | 1.05 |

Measurements: Individual broiler weights and feed intake was collected in order to calculate body weight and body weight gain, feed intake, and gain-to-feed ratio by phase.

Results. Table 2 shows the performance effect of broiler chicks when Peptiva® and MOS is incorporated in their diets.

TABLE 2
Growth performance of broiler chicks on two phases: d 0-14 and d 14-28

| | Product | Dose | BW (g) | BWG (g) | FI (g) | FCR |
|---|---|---|---|---|---|---|
| Day 0-14 | Avilamycin | 45 ppm | 442 | 402 | 583 | 1.453 |
| | Fish | 50 ppm | 409 | 369 | 555 | 1.505 |
| | hydrolysate + | 100 ppm | 417 | 377 | 541 | 1.433 |
| | MOS | 200 ppm | 427 | 388 | 552 | 1.417 |
| | (80:20) | | | | | |
| | SEM | | 9.836 | 9.769 | 21.933 | 0.060 |
| | P-value | | 0.1170 | 0.1034 | 0.9764 | 0.8156 |
| Day 14-28 | Avilamycin | 45 ppm | 1,550 | 1,108 | 1,628 | 1.542 |
| | Fish | 50 ppm | 1,459 | 1,051 | 1,578 | 1.544 |
| | hydrolysate + | 100 ppm | 1,431 | 1,015 | 1,596 | 1.621 |
| | MOS | 200 ppm | 1,453 | 1,026 | 1,624 | 1.620 |
| | (80:20) | | | | | |
| | SEM | | 43.63 | 42.35 | 37.47 | 0.0540 |
| | P-value | | 0.5313 | 0.7625 | 0.8733 | 0.8246 |

BW = body weight.
BWG = body weight gain.
FI = feed intake.
FCR = food conversion ratio.
SEM = Standard error mean.

Table 3 shows the growth performance of broiler chicks when Peptiva®+MOS is incorporated in their diets.

TABLE 3
Broiler chick growth performance days 0-28

| | Product | Dose | BW (g) | BWG (g) | FI (g) | FCR |
|---|---|---|---|---|---|---|
| Growth | Avilamycin | 45 ppm | 1,550 | 1,510 | 2,212 | 1.472 |
| performance | Fish | 50 ppm | 1,468 | 1,428 | 2,178 | 1.541 |
| Day 0-28 | hydrolysate + | 100 ppm | 1,437 | 1,397 | 2,224 | 1.593 |
| | MOS | 200 ppm | 1,444 | 1,404 | 2,116 | 1.508 |
| | (80:20) | | | | | |
| | SEM | | 43.63 | 43.34 | 52.78 | 0.0417 |
| | P-value | | 0.5313 | 0.5309 | 0.5519 | 0.7174 |

BW = body weight
BWG = body weight gain.
FI = feed intake.
FCR = food conversion ratio.
SEM = Standard error mean.

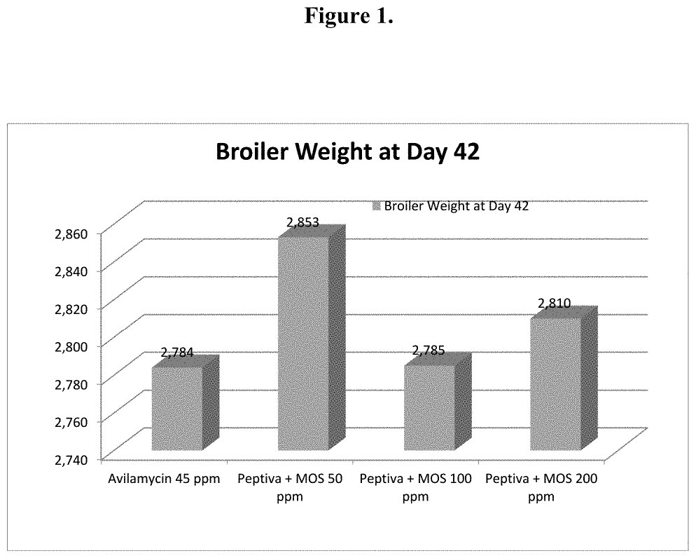

FIG. 1 and Table 4 show the growth performance of broiler chicks during the second half of the study when Peptiva®+MOS is incorporated in their diets.

TABLE 4
Broiler chick growth performance for days 28-42

| Growth | | | | | | |
|---|---|---|---|---|---|---|
| performance | | | | | | |
| d 28-42 | Product | Dose | BW (g) | BWG (g) | FI (g) | FCR |
| D 28-42 | Avilamycin | 45 ppm | 2,784 | 1,235 | 2,601 | 2.123 |
| | Fish | 50 ppm | 2,853 | 1,386 | 2,634 | 1.923 |
| | hydrolysate + | 100 ppm | 2,785 | 1,348 | 2,700 | 2.019 |
| | MOS (80:20) | 200 ppm | 2,810 | 1,367 | 2,632 | 1.929 |
| | Yeast | 50 ppm | 2,737 | 1,277 | 2,615 | 2.053 |
| | | 100 ppm | 2,779 | 1,348 | 2,695 | 2.010 |
| | | 200 ppm | 2,866 | 1,413 | 2,710 | 1.929 |
| | Fish | 50 + 50 ppm | 2,858 | 1,330 | 2,724 | 2.062 |
| | hydrolysate + | | | | | |
| | MOS (80:20) + | | | | | |
| | Yeast | | | | | |
| | SEM | | 35.62 | 48.46 | 47.42 | 0.0736 |
| | P-value | | 0.1990 | 0.2433 | 0.4628 | 0.5155 |

BW = body weight.
BWG = body weight gain.
FI = feed intake.
FCR = food conversion ratio.
SEM = Standard error mean.

Table 5 shows the overall growth performance of broiler chicks when Peptiva®+MOS is incorporated in their diets.

TABLE 5
Overall growth performance of broiler chicks (0-42 days)

| Product | Dose | BW (g) | BWG(g) | FI (g) | FCR |
|---|---|---|---|---|---|
| Avilamycin | 45 ppm | 2,784 | 2,745 | 4,813 | 1.753 |
| Fish | 50 ppm | 2,853 | 2,814 | 4,812 | 1.711 |
| hydrolysate + | 100 ppm | 2,785 | 2,746 | 4,924 | 1.793 |
| MOS (80:20) | 200 ppm | 2,810 | 2,771 | 4,748 | 1.714 |
| Yeast | 50 ppm | 2,737 | 2,697 | 4,748 | 1.761 |
| | 100 ppm | 2,779 | 2,739 | 4,801 | 1.754 |
| | 200 ppm | 2,866 | 2,826 | 4,886 | 1.730 |
| Fish | 50 + 50 ppm | 2,858 | 2,819 | 4,981 | 1.769 |
| hydrolysate + | | | | | |
| MOS (80:20) + | | | | | |
| Yeast | | | | | |
| SEM | | 35.62 | 35.64 | 77.12 | 0.0248 |
| P-value | | 0.1990 | 0.1958 | 0.4597 | 0.3921 |

BW = body weight.
BWG = body weight gain.
FI = feed intake.
FCR = food conversion ratio.
SEM = Standard error mean.

One-way ANOVA and Duncan's multiple range test was used to separate the means. There is no difference found between treatments. The overall mortality during the 42 days period is 3.65% and it is non-treatment specific.

Table 6 shows the bacterial counts of broiler chicks when Peptiva®+MOS is incorporated in their diets.

TABLE 6
Bacterial counts (Log₁₀ CFU/g) in the cecal samples
of the treated and control broiler chicks

| | | E. coli Counts | Lactobacilli Counts |
|---|---|---|---|
| Product | Dose | (Log₁₀ CFU/g) | (Log₁₀ CFU/g) |
| Avilamycin | 45 ppm | 7.29a | 7.21 |
| Fish | 50 ppm | 6.65ab | 7.17 |
| hydrolysate + | 100 ppm | 6.30ab | 7.44 |
| MOS (80:20) | 200 ppm | 6.18b | 7.18 |
| SEM | | 0.2369 | 0.1797 |
| P-value | | 0.0872 | 0.4133 |

¹ Means with different superscripts (letters) show a trend of difference at P < 0.1.
² All treatments are negative for Salmonella spp.

There is a trend of avilamycin having higher E. coli counts. There was no difference found in Lactobacilli counts. All treatments were negative for Salmonella spp.

### Example 2. Impact of Combining Fish Hydrolysate Combined with MOS in AGP-Free Diets Verses Standard Diets in Laying Hens

This study evaluated the impact of fish hydrolysate combined with mannan oligosaccharides (MOS) applied to diets free of AGP, upon the production efficiency of HY Line Brown laying hens during peak lay.

Further, the study analyzed the productive parameters (percent lay, feed intake, mortality) of peak lay laying hens (20-30 weeks) fed AGP-free diets supplemented with AGP free diet supplemented with fish hydrolysate combined with MOS, compared against conventional diets. The study also compared the internal and external egg quality variables (egg weight, shell and yolk color, albumen height, inclusions, yolk, albumen and shell weight, and shell thickness) from peak lay laying hens (20-30 weeks) fed AGP-free diets supplemented with fish hydrolysate combined with MOS, compared against conventional diets. The study also determined the effect of MOS supplementation on nutrient (MS, protein, and energy) digestibility, intestinal morphometry and microbiology from peak lay laying hens (20-30 weeks) fed AGP-free diets supplemented with fish hydrolysate combined with MOS, compared against conventional diets.

Materials and Methods. Location and housing: The experiment was carried out at the “El Salado” experimental farm belonging to the Universidad Cooperativa of Colombia, located at Ibagué city, Tolima Department, with coordinates 3° 24″N 74° 56″. This Unit sits in a place with a thermal range 19° C. to 31° C., at 1045 m.a.s.l. and 70% of average relative humidity. Birds were housed in an open house within traditional individual cages (30 cm×20 cm×40 cm, L×W×H) of pyramidal shape in a three-tier system. Altogether, 240 cages constituted the experimental population. The experimental unit was a set of three individual cages sharing a single feeder. Animals and trial duration: A total of 80 experimental Units were used during the trial. 2 initial weeks did serve as adaptation and training; the experimental phase lasted 8 weeks. The genetic line was Hy-Line Brown, brown shelled eggs type laying hen. Diets: Two corn-soybean diets adapted to peak lay hens were proposed. The only difference between them will be the presence (positive control) or not (negative control) of AGP. From the AGP-free diet three experimental diets were created for the testing of MOS, at doses of 100, 200, and 400 ppm. Therefore, the final design will be one of Randomized Complete Design (RCD). The composition of the diets and their nutritional analysis are set into Table 7.

TABLE 7
Ingredient composition and nutrients of experimental diets

| | Positive | Negative |
|---|---|---|
| Ingredients | Control (PC) | Control (NC) |
| Yellow corn | 53.600 | 53.600 |
| SBM 48% | 18.663 | 18.663 |
| Palm oil | 3.946 | 3.946 |
| Corn gluten meal | 3.000 | 3.000 |
| DCP | 1.817 | 1.817 |
| Wheat bran | 8.509 | 8.509 |
| Limestone, fine | 4.492 | 4.492 |
| Limestone, coarse | 4.000 | 4.000 |
| Vit-Min premix | 0.200 | 0.200 |
| DL- methionine | 0.183 | 0.183 |
| L-lysine HCl | 0.147 | 0.147 |
| Salt | 0.250 | 0.250 |
| Sodium bicarbonate | 0.100 | 0.100 |
| Choline chloride 60% | 0.052 | 0.052 |
| L-threonine | 0.028 | 0.028 |
| Enramycin 8% | 0.013 | 0.000 |
| Fish hydrolysate + MOS 100 gm | 0.000 | 0.010 |
| Fish hydrolysate + MOS 200 gm | 0.000 | 0.020 |
| Fish hydrolysate + MOS 400 gm | 0.000 | 0.040 |
| Celite ® | 0.040 | 0.040 |
| Celite ® | 1.000 | 1.000 |
| TOTAL | 100.0 | 100.1 |

| Nutrients | CP |
|---|---|
| AME, kcal/kg | 2800 |
| Crude protein, % | 16.00 |
| Ether extract, % | 6.157 |
| Crude fiber, % | 2.608 |
| Ash, % | 13.296 |
| Starch, % | 36.878 |
| Lysine, % | 0.75 |
| Methionine, % | 0.418 |
| Methionine + Cysteine, % | 0.65 |
| Threonine, % | 0.53 |
| Tryptophan, % | 0.177 |
| Arginine, % | 0.904 |
| Valine, % | 0.653 |
| Isoleucine, % | 0.592 |
| Linoleic acid, % | 1.50 |
| Av. phosphorus, % | 0.41 |
| Calcium, % | 3.70 |
| Na, % | 0.16 |
| K, % | 0.687 |
| Cl, % | 0.228 |
| Electrolyte balance, mEq/kg | 181.02 |

Table 7 has two different Celite® entries because the lower value compensates for the variable dose inclusion of the additive in the final formula, so the diet composition is matched gram by gram. The overall 1% Celite® was held constant in all diets but includes as a source of acid indigestible ash, which is used for nutrient digestibility analyses using intestinal contents as subject.

After a stabilization period, all hens in the experiment received orally a 10× the normal dose of coccidia vaccine Fortegra MSD, (containing oocysts of E. acervulina, E. maxima, E. maxima MFP, E. mivate and E. tenella). This treatment was intended to create an intestinal microbial dysbiosis which would be controlled or not by the AGP from the control diet and/or the experimental treatments.

Feeding and Handling: Feed was offered daily in fixed amounts, gradually increasing from 95 to 110 g/day, according to the management guide from the Hy Line manual, for the period from weeks 20 to 30. All hens received the control diet during the two first weeks of the trial (stabilization and training period). Water was offered ad libitum throughout the experiment, with the same conditions for all treatments. To compensate for the high pH in the water from local wells, an acidifier was added to it from the first day of the trial, so that the water going to the hens will have a pH lower than 6,5. The experimental diets were manufactured by Alcon de Colombia feed company and brought to the site from Buga (Valle Department).

Evaluation Parameters. Productive parameters: Feed consumption was recorded daily by weighing residuals from the day before offering. Lay rate was calculated daily, as a ratio from the number of birds and the number of eggs laid on the day. Eggs were weighed individually every day. Mortality and environment parameters were recorded first at the beginning of every working day. Intestinal microbiology: At the end of the experiment, one animal per replication was euthanized, the intestinal content from the Meckel diverticulum to the ileocecal junction was carefully and aseptically removed, and the contents were pooled, placed into Petri dishes and frozen at −20° C. until analysis. (Jahanian and Golshadi, Livestock Science 178 (June 2015)). Microbiology analysis were made in duplicate, according to the method reported by Baurhoo, B., et al., Poult Sci. 86: 1070 (2007). The ileal contents were serially diluted into sterile 0,85% saline, further seeded into McConkey Agar®, Lactobacillus MRS Agar®, Rapid E. coli 2 Agar®, Salmonella-Shigella Agar® for incubation at 37° C. for 48 hours, further evaluating coliforms, lactobacilli, E. coli, and Salmonella, respectively. Nutrient digestibility: Ileal nutrient digestibility analysis will be made taking into account that all diets contain a source of indigestible ash (Celite®). Intestinal morphometry: After careful collection of intestinal contents, 2 cm sections of jejunum were made. They were washed with saline, fixed by buffered formaldehyde until proceeding to histology, using HE tincture. The slides were measured using a Zeiss® microscope, with 10 measurements by bird, for villi height and crypt depth. Egg parameters: Analysis of internal and external egg quality parameters was performed every two weeks, whereas 18 eggs per treatment were collected, for a grand total of 72 eggs per period. Eggs were identified by treatment and replication and the following measurements were performed the day of collection:
Individual weight using a Vibra® scale with 0.001 g accuracy.
Shell color, using the HyLine® color rule.
Yolk color, using the Roche® color fan.
Albumen height, whit a 0.01 mm accuracy micrometer. From these parameters, Haugh Units (HU) were computed.
Yolk weight using a Vibra® scale with 0.001 g accuracy.
Shell weight: clean shells are stabilized at ambient temperature for 48 hours and further weighed using a Vibra® scale with 0.001 g accuracy.
Percent yolk and shell are computed against the total egg weight.
Albumen percent is computed as 100−(% yolk+% shell).
Shell thickness measured by 0.01 mm precision micrometer from 2 points in the shell (equator and the broad pole).

Statistical analysis: All data were subject to statistical analysis by means of the SPSS package. Generally speaking, GLM with ANOVA was performed and if after testing the Levene's statistics is not significant, then the means will be separated by LSD, Duncan or Tukey. When Levene's statistics were significant, the data was analyzed by non-parametric procedure Kruskal-Wallis and Dunnett's T3 tests. All declarations of significance were made with p<0.05.

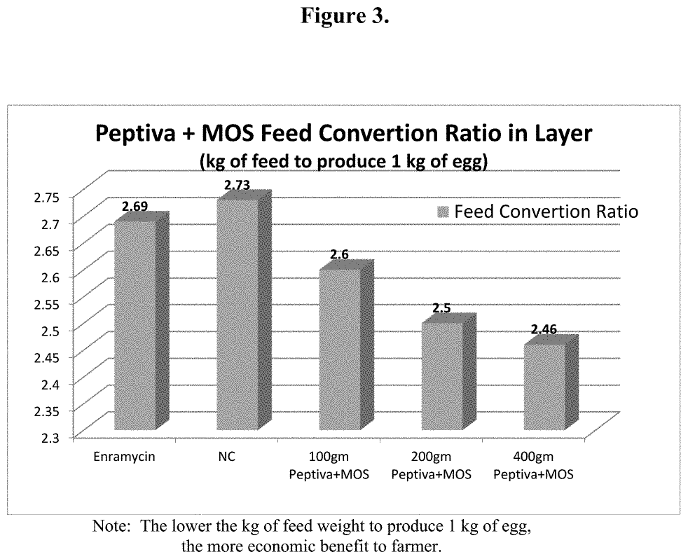

Results. FIG. 3 and Table 8 show that there were not any detectable differences between the positive control and the three MOS treatments in lay rate. The negative control was significantly lower than all other treatments. Further, feed intake from MOS treatments was significantly higher than both the negative and positive controls. The egg weight from MOS400 treatment was higher and significantly different from the negative control, but not from the other MOS treatments and the positive control. Egg FCR from MOS400 and MOS 200 were lower and significantly different from MOS100, the positive control and negative control.

TABLE 8
Overall parameters from the experiment with MOS as AGP replacers

| | | | Feed intake, | | | | | |
|---|---|---|---|---|---|---|---|---|
| | Lay rate, % | | g/hen/day | | Avg egg weight, g | | FCR egg, g | |
| | Mean | SD | Mean | SD | Mean | SD | Mean | SD |
| Negative Control (NC) | 70.90^{b} | 27.924 | 92.75^{c} | 13.436 | 56.33^{bc} | 25.185 | 2.73^{b} | 1.666 |
| Positive Control (PC) | 80.62^{a} | 23.146 | 99.84^{b} | 12.716 | 57.21^{ac} | 25.058 | 2.69^{b} | 4.281 |
| Fish Hydro + MOS100 | 79.92^{a} | 23.625 | 102.07^{a} | 9.023 | 58.53^{ac} | 37.284 | 2.60^{ab} | 1.432 |
| Fish Hydro + MOS200 | 79.80^{a} | 26.830 | 101.49^{a} | 9.004 | 57.79^{ac} | 26.189 | 2.50^{a} | 1.830 |
| Fish Hydro + MOS400 | 79.66^{a} | 25.897 | 101.92^{a} | 8.420 | 59.10^{a} | 32.071 | 2.46^{a} | 1.167 |

FCR = Feed Conversion Ratio (feed consumed to gain 1 kg of egg).
Superscripts a-c indicate p < 0.05, which is significant

Tables 9 to 11 show the weekly values for the analyzed parameters from all treatments in the experiment. Table 9 shows that the intake values in general peaked around week 29 and somewhat decreased thereafter. Feed intake from the negative control was consistently lower than any other treatments throughout the experiment. The MOS treatments achieved higher feed intake than either the negative or positive control for most of the 8 weeks of the duration of the experiment. MOS100 has the highest mean intake at the end of the experiment.

TABLE 9
Average weekly feed intake (g/day) for all treatments with MOS as AGP replacers

| | Wk. 23 | Wk. 24 | Wk. 25 | Wk. 26 | Wk. 27 | Wk. 28 | Wk. 29 | Wk. 30 | Wk. 31 | Mean |
|---|---|---|---|---|---|---|---|---|---|---|
| NC | 94.93^{b} | 94.43^{c} | 87.78^{b} | 88.89^{b} | 93.85^{b} | 92.50^{b} | 97.74^{c} | 99.36^{b} | 85.58^{c} | 92.78 |
| PC | 99.73^{a} | 100.79^{ab} | 99.05^{a} | 100.39^{a} | 101.18^{a} | 101.12^{a} | 103.41^{b} | 101.16^{b} | 92.12^{b} | 99.88 |
| FH + MOS 100 | 101.80^{a} | 100.30^{b} | 102.37^{a} | 98.03^{a} | 100.77^{a} | 102.09^{a} | 105.81^{a} | 104.42^{a} | 102.66^{a} | 102.03 |
| FH + MOS 200 | 98.35^{a} | 99.65^{b} | 99.82^{a} | 99.88^{a} | 103.98^{a} | 102.78^{a} | 105.59^{a} | 103.29^{a} | 100.79^{a} | 101.57 |
| FH + MOS 400 | 101.80^{a} | 102.73^{ab} | 102.79^{a} | 101.12^{a} | 102.18^{a} | 99.51^{a} | 102.66^{b} | 102.41^{a} | 102.19^{a} | 101.93 |

NC = Negative Control, PC = Positive Control, FH = fish hydrolysate.
Superscripts a-c indicate p < 0.05, which is significant

Table 10 shows that lay rates peaked around week 29 and somewhat decreased thereafter. The lay rate from the negative control was consistently lower than any other treatments throughout the experiment. MOS treatments achieved similar lay rates than the positive control at the end of the 8 weeks of the experiment, although weekly values were more variable than values from feed intake between groups. MOS200 is the treatment with the nearest lay rate to that of the positive control diet.

TABLE 10
Average weekly lay rate (%) for all treatments with MOS as AGP replacers

| | Wk. 23 | Wk. 24 | Wk. 25 | Wk. 26 | Wk. 27 | Wk. 28 | Wk. 29 | Wk. 30 | Wk. 31 | Mean |
|---|---|---|---|---|---|---|---|---|---|---|
| NC | 51.49^{b} | 62.50 | 69.94^{b} | 70.83^{c} | 74.58^{bc} | 80.06^{b} | 79.17^{c} | 80.06^{c} | 70.54^{b} | 71.02 |
| PC | 67.94^{a} | 71.43 | 85.40^{a} | 85.71^{a} | 87.56^{ab} | 86.98^{b} | 84.76^{bc} | 84.13^{b} | 73.65^{b} | 80.84 |
| FH + MOS100 | 61.31^{a} | 66.96 | 77.98^{ab} | 81.25^{b} | 79.17^{bc} | 86.90^{b} | 90.18^{ab} | 90.48ab | 84.82^{a} | 79.89 |
| FH + MOS200 | 54.72^{a} | 64.71 | 76.47^{b} | 75.07^{bc} | 89.41^{ab} | 91.60^{a} | 92.44^{a} | 93.00^{a} | 83.75^{a} | 80.13 |
| FH + MOS400 | 57.66^{a} | 67.86 | 80.95^{a} | 86.31^{a} | 84.58^{b} | 90.18^{a} | 87.50^{ab} | 84.52^{b} | 78.57^{ab} | 79.79 |

NC = Negative Control, PC = Positive Control, FH = fish hydrolysate.
Superscripts a-c indicate p < 0.05, which is significant

As shown in Table 11, computing the total egg mass by week, we find that MOS200 obtains the highest egg mass value, followed by the other two MOS treatments. The values are higher than either the negative control or the positive control diets. At the limit, we could say that MOS treatments compensates for the removal of the AGP from the diets.

TABLE 11
Weekly egg mass (g) for all treatments in the experiment with MOS as AGP replacers

| | Wk. 23 | Wk. 24 | Wk. 25 | Wk. 26 | Wk. 27 | Wk. 28 | Wk. 29 | Wk. 30 | Wk. 31 | Total, kg |
|---|---|---|---|---|---|---|---|---|---|---|
| NC | 9.145 | 11.405 | 12.959 | 13.093 | 9.916 | 16.745 | 15.051 | 16.633 | 12.653 | 117.6 |
| PC | 11.454 | 12.398 | 17.513 | 15.146 | 11.287 | 15.658 | 16.257 | 15.465 | 12.625 | 127.8 |
| FH + MOS100 | 10.856 | 14.006 | 16.093 | 15.309 | 13.777 | 16.594 | 18.855 | 17.198 | 16.080 | 138.8 |
| FH + MOS200 | 10.453 | 12.670 | 15.200 | 16.752 | 13.006 | 18.710 | 20.859 | 19.074 | 17.935 | 144.7 |
| FH + MOS400 | 10.303 | 13.450 | 17.539 | 16.360 | 13.290 | 19.131 | 17.191 | 16.387 | 15.121 | 138.8 |

NC = Negative Control,
PC = Positive Control,
FH = fish hydrolysate

Egg quality parameters. The following Tables show the various parameters tested from 4 collections of eggs (Coll. 1 to Coll. 4) from all treatments, taken at two-week intervals. Table 12 shows the weight of eggs, in grams, and the Haugh Units from sample eggs collected at two-week intervals from all treatments during the experiment.

TABLE 12
Egg weight (g) and Haugh Units in eggs from all treatments with MOS as AGP replacers

| | Egg weight, g | | | | Haugh Units | | | |
|---|---|---|---|---|---|---|---|---|
| | Coll. 1 | Coll. 2 | Coll. 3 | Coll. 4 | Coll. 1 | Coll. 2 | Coll. 3 | Coll. 4 |
| NC | 54.72^{b} | 54.83^{b} | 55.78^{b} | 53.68^{b} | 102.18^{a} | 93.21 | 99.30 | 101.45 |
| PC | 56.68^{ab} | 56.74^{ab} | 58.79^{ab} | 56.52^{b} | 97.47^{ab} | 91.73 | 98.75 | 100.20 |
| FH + MOS100 | 55.94^{ab} | 56.05^{ab} | 58.18^{ab} | 56.56^{b} | 96.49^{b} | 91.52 | 99.13 | 101.70 |
| FH + MOS200 | 54.11^{ab} | 54.97^{b} | 56.93^{ab} | 56.35^{b} | 96.42^{b} | 94.39 | 100.16 | 100.85 |
| FH + MOS400 | 55.73^{a} | 58.61^{a} | 59.78^{a} | 60.27^{a} | 99.81^{ab} | 92.83 | 100.40 | 102.71 |

NC = Negative Control, PC = Positive Control, FH = fish hydrolysate.
Superscripts a-c indicate p < 0.05, which is significant

As for egg weight from the samples, those from the negative control diets were lower than all other diets in most collections. Diet MOS400 did show a time-related increase in egg weight from collection 1 to collection 4, being significantly heavier than the negative control and significantly heavier than all other diets at collection 4, the last one taken in the experiment.

With the exception of some treatments from collection 1, Haugh Units did not vary among treatments for the remaining collections.

Table 13 shows the percentage of shell, yolk, and egg white from sample eggs collected at two-week intervals from all treatments during the experiment.

TABLE 13
Percent of shell, yolk, and egg white in eggs from all treatments with MOS as AGP replacers

| | Shell, % | | | | Yolk, % | | | | Egg white, % | | | |
|---|---|---|---|---|---|---|---|---|---|---|---|---|
| | Coll. 1 | Coll. 2 | Coll. 3 | Coll. 4 | Coll. 1 | Coll. 2 | Coll. 3 | Coll. 4 | Coll. 1 | Coll. 2 | Coll. 3 | Coll. 4 |
| NC | 10.28 | 9.38^{b} | 10.03 | 9.97 | 22.99 | 24.59^{a} | 24.03^{a} | 24.58^{a} | 66.74 | 66.03^{b} | 65.93 | 65.44 |
| PC | 10.41 | 10.46^{a} | 10.45 | 10.21 | 21.83 | 22.34^{ab} | 22.44^{b} | 22.92^{b} | 67.74 | 67.89^{a} | 67.11 | 66.88 |
| FH + MOS 100 | 10.47 | 10.29^{a} | 10.20 | 9.88 | 22.83 | 23.61^{a} | 23.91^{a} | 24.51^{a} | 66.69 | 66.18^{b} | 65.89 | 65.62 |
| FH + MOS 200 | 10.61 | 10.52^{a} | 10.43 | 9.41 | 22.59 | 23.58^{a} | 23.39^{ab} | 23.92^{ab} | 66.81 | 66.21^{b} | 66.19 | 66.68 |
| FH + MOS 400 | 10.45 | 10.21^{a} | 10.04 | 9.52 | 22.21 | 21.23^{b} | 23.33^{ab} | 23.68^{ab} | 67.35 | 68.58^{a} | 66.62 | 66.81 |

NC = Negative Control, PC = Positive Control, FH = fish hydrolysate.
Superscripts a-c indicate p < 0.05, which is significant

Percent of shell, yolk and egg white from all treatments did show small variations that in some cases were statistically significant, although a fair observation may be that the variations of net values were small throughout the experiment.

Table 14 shows egg white height, and eggshell thickness at the equator and the broadest pole.

TABLE 14
Egg white height (mm), and eggshell thickness (μm) in eggs from all treatments in with MOS as AGP replacers

| | Egg white height, mm | | | | Equator thickness, μm | | | | Broad pole thickness, μm | | | |
|---|---|---|---|---|---|---|---|---|---|---|---|---|
| | Coll. 1 | Coll. 2 | Coll. 3 | Coll. 4 | Coll. 1 | Coll. 2 | Coll. 3 | Coll. 4 | Coll. 1 | Coll. 2 | Coll. 3 | Coll. 4 |
| NC | 10.54^{a} | 8.70 | 9.86 | 10.21 | 439^{b} | 394^{b} | 407 | 381 | 403 | 346^{b} | 360 | 343 |
| PC | 9.54^{ab} | 8.35 | 9.82 | 10.07 | 474^{a} | 425^{a} | 414 | 402 | 420 | 390^{a} | 382 | 366 |
| FH + MOS 100 | 9.42^{ab} | 8.35 | 9.90 | 10.38 | 454^{ab} | 427^{a} | 407 | 386 | 409 | 387^{a} | 374 | 349 |
| FH + MOS 200 | 9.12^{b} | 8.75 | 10.07 | 10.19 | 443^{ab} | 427^{a} | 419 | 381 | 409 | 395^{a} | 377 | 338 |
| FH + MOS 400 | 10.08^{ab} | 8.67 | 10.25 | 10.86 | 449^{ab} | 445^{a} | 411 | 381 | 409 | 401^{a} | 371 | 333 |

NC = Negative Control, PC = Positive Control, FH = fish hydrolysate.
Superscripts a-c indicate p < 0.05, which is significant

Table 15 shows the values for shell and yolk white from sample eggs collected at two-week intervals from all treatments during the experiment.

TABLE 15
Shell and yolk color values in eggs from all treatments with MOS as AGP replacers

| | Shell color | | | | Yolk color | | | |
|---|---|---|---|---|---|---|---|---|
| | Coll. 1 | Coll. 2 | Coll. 3 | Coll. 4 | Coll. 1 | Coll. 2 | Coll. 3 | Coll. 4 |
| NC | 92.50 | 86.25 | 94.17 | 92.14 | 11.63^{a} | 10.00^{b} | 9.92^{b} | 10.14^{a} |
| PC | 91.88 | 90.00 | 91.33 | 86.67 | 11.13^{a} | 10.62^{ab} | 10.87^{a} | 10.00^{a} |
| FH + MOS100 | 94.38 | 91.33 | 91.25 | 88.13 | 11.75^{a} | 10.67^{ab} | 11.13^{a} | 10.13^{a} |
| FH + MOS200 | 89.33 | 90.62 | 94.00 | 90.67 | 10.67^{b} | 10.50^{ab} | 10.73^{a} | 9.33^{b} |
| FH + MOS400 | 90.00 | 92.50 | 91.54 | 90.77 | 11.60^{a} | 11.19^{a} | 11.08^{a} | 10.62^{a} |

NC = Negative Control, PC = Positive Control, FH = fish hydrolysate.
Superscripts a-c indicate p < 0.05, which is significant

There were no statistically significant differences in shell color values. As for yolk, the values were mostly homogeneous, although MOS200 had some values lower than other treatments, as did the negative control diet. Data for inclusions was analyzed by Chi square evaluating meat type inclusions, blood type inclusions, and no inclusions. Pearson Chi-squared correlation between diet and inclusions was not significant (p=0.104), which allows us to say that there was no difference between treatments on this parameter.

Intestinal content microbiology. Table 16 Culture microbiology of pooled intestinal content was performed. Table 16 contains the values obtained from all treatments in the experiment.

TABLE 16
Microbial counts (log) from all treatments
with MOS as AGP replacers

| | Lactobacilli | E. coli | Bifidobacterium | Salmonella |
|---|---|---|---|---|
| NC | 6,331 | 5,228 | 6,913 | Negative |
| PC | 7,186 | 4,684 | 6,442 | Negative |
| FH + MOS100 | 6,706 | 5,267 | 5,738 | Negative |
| FH + MOS200 | 7,242 | 5,349 | 7,293 | Negative |
| FH + MOS400 | 7,073 | 4,694 | 6,843 | Negative |

NC = Negative Control,
PC = Positive Control,
FH = fish hydrolysate

The differences in log count of species from intestinal content of all diets in the experiment gave non-significant differences. Apparently, the treatments did not influence the microbial make-up of the animals on test, although this can be construed as hens being much more mature individuals than broiler chickens, with a well stablished flora that is difficult to change.

Conclusions. The use of fish peptide+MOS products, as tested in this experiment has shown the ability to maintain hen performance, egg productivity and egg quality when AGP are removed from the diet of laying hens, under the condition used for this experiment. This Example confirms the potential value of Fish Hydrolysate combined with MOS as an AGP substitute and fit into the present trend in modern animal nutrition of reducing or altogether suppressing the use of sub-therapeutic antibiotics in production animals.

### Example 3. Effects Feeding Diets Containing FH Combined with MOS to Broiler Chickens

This example was aimed to compare the productive performances, after 42 days, of broiler chickens fed a standard control diet, an AGP-deprived diet, and AGP-deprived diets containing fish hydrolysate (FH) and mannan oligosaccharide (MOS); fed alone or in combination. The diets are described below, and their compositions are shown in Tables 17-19. Additionally, a set of samples for procedures related to intestinal health, ingredient digestibility, and intestinal microbiology were taken for further analysis.

Materials and Methods. The experiment was run in floor pens in the joint Tekzol-University of Tolima Poultry Experimental Unit located at Armero, Colombia. The rearing house has 64 pens, 3.4 m² each, holding 42 chickens with an initial density of 12.3 birds/m² and a final meat load of 35 kg/m². Birds were reared in clean and disinfected pens after a proper sanitary void before the start of the test. A layer of 10-15 cm of new rice husks was used as litter; it was disinfected with quaternary ammonia and glutaraldehyde. Feeders were all identical, all of the same kind, size, color and any other visible characteristic. Each pen was identified with a lettered mark indicating the pen number and kind of feed received. Chickens had feed and water available ad libitum and feed consumption was measured. The lighting program was 24 hours a day for the three first days and further decreasing artificial lightning by 4 hours a day until operating in a natural light/dark cycle from 6 days onwards. With every diet change, the feeders were emptied, the remains, if any, weighed, and feeders refilled with fresh feed. This process was carried out very carefully to avoid feed spilling.

General appearance of the flock, temperature, light, water, feed, litter condition and mortality were monitored and registered on a daily basis. Animals with very poor performance were removed from the trial, and the date of their removal and their weight was recorded for data adjustment.

One-day old, male Ross 308 broiler chickens were obtained from a local supplier and used for the trial. The chickens were distributed at random into treatments and replicates, with pen numbers allocation done by using the RANDOM function of SPSS. The trials design conforms to a completely randomized design, with 7 replicates per treatment. Within all replicates, 15 birds were numbered with an individual tag and were weighed at every weighing point. This serves as internal contrast and provides insight on pen homogeneity.

Two corn-soya bases diets in meal form are proposed from which 5 treatments were created. The feeding program had three phases: the pre-starter phase (I), from 1 to 10 days; the starter phase (II), from 11 to 22 days of age; and the grower phase (III) from 23 to 42 days. All diets were fed ad libitum and two samples were taken of each type of concentrate and each batch and made available to Tekzol for bromatology studies. Feed composition is shown in Table 17 and the treatments are set in Table 18.

On the 5th day of the experiment, the litter of all pens were sprayed with an oral coccidia vaccine (MSD Fortegra, containing oocysts of E. acervulina, E. maxima, E. maxima MFP, E. mivate and E. tenella) at 8× times the regular dose. The objective for this challenge was creating microbial intestinal conditions leading to disbacteriosis in the chicks, which will be controlled or not by the treatments. Together with performance parameters, lesions and gut health this indicates success or failure of the treatments.

Performance Evaluations. On day 21 and 42, 2 chicks from every replication were euthanized and necropsy performed. Vents and footpads were evaluated. The intestine was evaluated for macroscopic lesions consistent with Eimeria invasion, other type of macroscopic lesions, and sections of intestine were taken for morphometry analysis, yielding macroscopic and microscopic data of gut integrity. Apparent consumption: Feed disappearance was recorded at diet changes. Average daily intake, gain and period feed conversion ratio were calculated per pen. The conversion ratio adjusted by mortality was calculated as follows: total feed intake per period and pen/(total live weight of the pen+weight of the dead birds per pen)−total live weight of the pen in a former period. Live weight: All animals were pool weighed according to the protocol followed in previous tests, at day 0 (initial), at diet changes at 11 and 22 days. Final weighing was at day 42, end of the experiment. All weights were taken using an electronic scale with ±1 g accuracy. Mortality was recorded and dead animals weighed to adjust the period performance taking into account this parameter. Records were kept during the experiment on the number of animals with thick stools or any other contingency. Statistical Analysis: The statistical program SPSS was used to carry out an analysis of variance (ANOVA) on the data obtained, to assess the effect of the experimental diets on chicken performance. Under normal circumstances, the general lineal model with univariate analysis of variance was used. All declarations of significance were based on a probability level of p<0.05. Duncan Multiple Range (DMR) test or Tukey were used to separate averages. If homogeneity of variance was not achieved using Levene, non-parametric tests (eg. Independ Samples Kruskal-Wallis test) were tried.

TABLE 17
Ingredients and nutritional composition
of basal diets used in Example 3

| Ingredients % | Pre starter % | Starter % | Grower % |
|---|---|---|---|
| Yellow corn | 57.061 | 57.967 | 57.061 |
| Soy bean meal 48% CP | 29.157 | 27.879 | 29.157 |
| Palm oil | 2.512 | 4.216 | 2.512 |
| Corn gluten meal | 4.000 | 4.000 | 4.000 |
| DCP | 2.096 | 1.848 | 2.096 |
| Hemoglobin | 2.500 | 1.500 | 2.500 |
| Limestone | 0.800 | 0.829 | 0.800 |
| Vit-min premix | 0.600 | 0.600 | 0.600 |
| DL- methionine | 0.381 | 0.329 | 0.381 |
| L-lysine HCl | 0.231 | 0.213 | 0.231 |
| Salt | 0.264 | 0.252 | 0.264 |
| Sodium bicarbonate | 0.200 | 0.200 | 0.200 |
| Choline chloride | 0.100 | 0.100 | 0.100 |
| L-threonine | 0.098 | 0.067 | 0.098 |
| AME, kcal/kg | 3,000 | 3,100 | 3,200 |
| Crude protein, % | 23.000 | 21.500 | 19.500 |
| Ether extract, % | 4.838 | 6.458 | 7.547 |
| Crude fiber, % | 2.444 | 2.409 | 2.320 |
| Ash, % | 6.672 | 6.349 | 5.856 |
| Starch, % | 37.364 | 37.948 | 40.247 |
| Lysine, % | 1.280 | 1.150 | 1.030 |
| Methionine, % | 0.708 | 0.639 | 0.584 |
| Methionine + Cysteine, % | 0.998 | 0.920 | 0.845 |
| Threonine, % | 0.870 | 0.782 | 0.711 |
| Tryptophan, % | 0.251 | 0.233 | 0.206 |
| Arginine, % | 1.268 | 1.200 | 1.072 |
| Valine, % | 1.068 | 0.961 | 0.854 |
| Isoleucine, % | 0.820 | 0.790 | 0.717 |
| Linoleic acid, % | 1.311 | 1.540 | 1.675 |
| Av. phosphorus, % | 0.480 | 0.440 | 0.390 |
| Calcium, % | 0.900 | 0.850 | 0.800 |
| Sodium, % | 0.200 | 0.190 | 0.170 |
| Potassium, % | 0.863 | 0.834 | 0.758 |
| Chloride, % | 0.274 | 0.257 | 0.241 |

TABLE 18
The diets design used as treatments in the trial

| Diet | | |
|---|---|---|
| Number | Diet Name | Contents |
| 1 | Positive Control (PC) | Basal Diet + Avilamycin 50 gm |
| 2 | Negative Control (NC) | Basal Diet |
| 3 | Treatment 1 FH + MOS50 | Basal Diet + fish hydrolysate |
| | | (FH)/MOS 50 gm |
| 4 | Treatment 2 FH + MOS100 | Basal Diet + fish hydrolysate |
| | | (FH)/MOS 100 gm |
| 5 | Treatment 3 FH + MOS200 | Basal Diet + fish hydrolysate |
| | | (FH)/MOS 2000 gm |

Results. Table 19 shows the body weight of the chicks when Peptiva® and MOS is incorporated in their diets.

TABLE 19
Body weight (Means and SD) of all treatments, grams (gm)

| | 5 days | | 11 days | | 22 days | | 42 days | |
|---|---|---|---|---|---|---|---|---|
| Diet | Mean | SD | Mean | SD | Mean | SD | Mean | SD |
| 1 | 92.50 | 2.60 | 250.61 | 9.89 | 911.45 ^{b} | 22.44 | 2864.48 ^{a} | 106.56 |
| 2 | 91.79 | 2.78 | 249.83 | 10.16 | 922.77 ^{b} | 30.61 | 2721.01 ^{b} | 55.63 |
| 3 | 92.14 | 3.59 | 246.70 | 3.77 | 919.18 ^{b} | 37.21 | 2766.75 ^{b} | 86.47 |
| 4 | 92.50 | 2.50 | 251.39 | 4.03 | 929.90 ^{b} | 15.68 | 2894.07 ^{a} | 198.49 |
| 5 | 90.00 | 4.08 | 249.06 | 12.18 | 898.07 ^{b} | 27.36 | 2765.96 ^{b} | 101.26 |

^{a, b, c} means with different superscripts are significantly different (p < 0.05)

At the end of the trial, diet 2 that had no antibiotic (AGP), had the worst weight gain and is significantly different than that of the diet 1, an AGP-positive diet and diet 4. Diet 4 (Treatment 4) reached highest body weight at the end of the experiment. Diet 4 (contains 100 grams (gm) of fish hydrolysate+MOS) had 6.36% better weight gain than Diet 2 (diet contained no AGP). Diet 4 had the best weight gain though out the trial and a strong trend over Diet 3 (p=0.058).

Table 20 shows the feed intake of the chicks when Peptiva® and MOS is incorporated in their diets.

TABLE 20
Feed intake for all treatments is shown in Table 4, (gm)

| | 11 Day | | 22 Day | | 42 Day | |
|---|---|---|---|---|---|---|
| Diet | Means | SD | Means | SD | Means | SD |
| Diet 1 | 204.99 ^{a} | 5.75 | 1236.44 | 37.94 | 4717.16 | 201.65 |
| Diet 2 | 203.49 ^{a} | 11.82 | 1310.00 | 148.28 | 4637.51 | 205.31 |
| Diet 3 | 189.65 ^{b} | 11.06 | 1262.34 | 67.68 | 4637.62 | 280.40 |
| Diet 4 | 198.49 ^{b} | 10.14 | 1279.38 | 55.85 | 4761.35 | 325.07 |
| Diet 5 | 203.28 ^{a} | 11.16 | 1322.47 | 165.25 | 4611.11 | 187.56 |

^{a, b} means with different superscripts are significantly different (p < 0.05)

With the exception of 11-day consumption, there were no significant differences between treatments in the cumulative feed intake values on day 42.

Table 21 shows the feed conversion ratio (FCR) of the chicks when Peptiva®+MOS is incorporated in their diets. There were no significant differences in FCR among groups.

TABLE 21
Feed Conversion Ratio (FCR) of the trial.

| | 11 Day | | 22 Day | | 42 Day | |
|---|---|---|---|---|---|---|
| Diet | Means | SD | Means | SD | Means | SD |
| Diet 1 | 0.819 | 0.025 | 1.357 | 0.055 | 1.647 | 0.035 |
| Diet 2 | 0.816 | 0.068 | 1.419 | 0.141 | 1.706 | 0.068 |
| Diet 3 | 0.769 | 0.044 | 1.377 | 0.121 | 1.676 | 0.079 |
| Diet 4 | 0.790 | 0.045 | 1.376 | 0.072 | 1.646 | 0.053 |
| Diet 5 | 0.818 | 0.064 | 1.475 | 0.198 | 1.668 | 0.052 |

Diet 4 (contains 100 gm of fish hydrolysate+MOS) had an average of 4.25% better FCR than Diet 2 (non-AGP group).

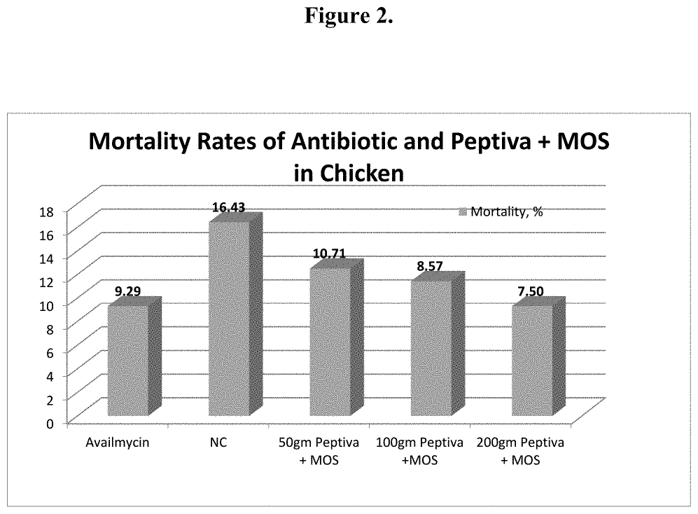

FIG. 2 and Table 22 show the mortality of the chicks when Peptiva®+MOS is incorporated in their diets.

TABLE 22
Mortality (percent) at day 42.

| Diet | Mean | SD |
|---|---|---|
| Diet 1 | 9.29 ^{b} | 4.01 |
| Diet 2 | 16.43 ^{ab} | 4.76 |
| Diet 3 | 10.71 ^{b} | 5.35 |
| Diet 4 | 8.57 ^{b} | 6.27 |
| Diet 5 | 7.50 ^{bc} | 6.29 |

^{a, b, c} means with different superscripts are significantly different (p < 0.05)

The highest mortality rate appeared on Treatment 2 (no AGP negative control diet), and this different was statistically significant (P<0.05) compares to Treatment 5. The remaining pairwise comparisons did not show signification at the level set by the analysis. The highest mortality seen in diet 2 reflects the impact of microbial disruption caused by the coccidia vaccine used at 8× the recommended dose and the absence of an antibiotic in that diet. Most other diets have numerically lower mortality rates, not reaching the level of significance, the difference between Diets 1 and 2, and Diets 2 and being just trends (p=0.064 in both comparisons). Diet 5 (contains 200 gm of fish hydrolysate+MOS), had mortality rate of 19.72% better than AGP diet, and 54.35% better than no AGP group and these differences are statistically significant.

Discussion and Conclusion. From a growth and intake standpoint, Diet 4 has performed as good as the AGP-containing Control diet (Diet 1) and Diet 4 (contains 100 gm of fish hydrolysate+MOS) had 6.36% better weight gain than Diet 2 (diet contained no AGP) and the difference is statistically significant.

Body weight, feed intake and mortality of such diets were identical to the AGP-control and significantly different from the negative control diet that lacked AGP. From the point of feed efficiency view, Diet 4 (contains 100 gm of fish hydrolysate+MOS) had an average of 4.25% better FCR than Diet 2 (non-AGP group). When comparing the mortality rates, AGP group and fish hydrolysate groups were all significantly better than non-AGP diet. Diet 5 (contains 200 gm of fish hydrolysate+MOS), had mortality rate of 19.72% better than AGP diet, and 54.35% better than non-AGP diet and these differences are statistically significant.

This Example demonstrates that the coccidia challenge is an experimental method that allows the researcher to create a microbial challenge in the birds, leading to impaired growth and increased mortality. This is one of the effects we were looking for in this phase of the experimentation because this simulated the real farms condition for chickens that are raised in. Coccidiosis is known to be a serious disease for the broiler and layer industries, especially in the currently consumers demands of “no antibiotic feed”, “no hormone feed” and “free range chicken”. This trial has demonstrated that fish hydrolysate when combined with MOS is better than the industry average just using MOS along. This combination product could be a solution to help poultry farmers to deliver safe meat and eggs that consumers want in an economically efficient way.

I claim:

1. A feed supplement composition comprising a fish-derived hydrolysate peptide (FHP) composition and mannan oligosaccharide (MOS) composition.

2. The feed supplement composition of claim 1, wherein the FHP composition and the MOS composition are each present in a ratio of 1:0.005 to 1:1 by weight.

3. The feed supplement composition of claim 1, wherein the feed supplement composition comprises 40-80% of protein.

4. The feed supplement composition of claim 1, wherein the feed supplement composition comprises 4-10% of fat.

5. The feed supplement composition of claim 1, wherein the feed supplement composition comprises 0.5-10% of fiber.

6. An animal food product comprising a feed supplement composition according to claim 1.

7. The animal food product of claim 6, wherein the feed supplement composition is 0.01-10% of the animal food product.

8. The animal food product of claim 7, wherein the feed supplement composition is 0.1-2.0% of the animal food product.

9. The animal food product of claim 8, further comprising corn, soy, fat, limestone powder, amino acids, and vitamins.

10. The animal food product of claim 6, wherein the animal food product does not comprise an antibiotic growth promoter (AGP).

11. A method of feeding poultry by supplying to the poultry a feed supplement composition according to claim 1.

12. A method of feeding poultry by supplying to the poultry an animal food product according to claim 6.

13. The method of claim 12, wherein the poultry comprise broiler chicks, grower chicks, finisher chicks, layer poults, egg laying hens, turkey poults, young turkeys, or grower-finisher turkeys.

14. The method of claim 13, wherein the poultry comprise broiler chicks; and
whereby supplying to the broiler chicks the animal food product results in an increase in average daily weight gain, an increase in body weight, an increase in average daily feed intake, an increase in the ratio of weight gain to feed, or any combination thereof, of the broiler chicks as compared to comparable broiler chicks fed only with a food product lacking a FHP and MOS.

15. The method of claim 13, wherein the poultry comprise broiler chicks; and
whereby supplying to the broiler chicks the animal food product results in a similar average daily weight gain, a similar increase in body weight, a similar average daily feed intake, a similar ratio of weight gain to feed, or any combination thereof, of the broiler chicks as compared to comparable broiler chicks fed with only with a food product comprising an antibiotic growth promoter (AGP) and lacking a FHP and MOS.

16. The method of claim 13, wherein the poultry comprise broiler chicks; and
whereby supplying to the broiler chicks the animal food product results in a decrease in mortality rate of the broiler chicks as compared to comparable boiler chicks fed with a food product lacking a FHP and MOS or the aforementioned food product comprising an antibiotic growth promoter (AGP).

17. The method of claim 13, wherein the poultry are egg laying hens; and
whereby supplying to the egg laying hens the animal food product results in an increase in feed intake of the egg laying hens as compared to comparable egg laying hens fed with a food product lacking a FHP and MOS.

18. The method of claim 13, wherein the poultry are egg laying hens; and
whereby supplying to the egg laying hens the animal food product results in an increase in feed intake of the egg laying hens as compared to comparable egg laying hens fed with a food product comprising an antibiotic growth promoter (AGP) and lacking a FHP and MOS.

19. The method of claim 13, wherein the poultry are egg laying hens; and
whereby supplying to the egg laying hens the animal food product results in an increase in egg weight, a decrease in egg feed conversion ratio, or a combination thereof of eggs laid by the egg laying hens as compared to eggs laid by comparable egg laying hens fed with a food product lacking a FHP and MOS.

20. The method of claim 13, wherein the poultry are egg laying hens; and
whereby supplying to the egg laying hens the animal food product results in an increase in egg weight, a decrease in egg feed conversion ratio, or a combination thereof of eggs laid by the egg laying hens as compared to eggs laid by comparable egg laying hens fed with a food product comprising an antibiotic growth promoter (AGP) and lacking a FHP and MOS.

21. The method of claim 13, wherein the poultry are egg laying hens; and
whereby supplying to the egg laying hens the animal food product results in a similar egg laying rate by the egg laying hens as compared to the egg laying rate by comparable egg laying hens fed with a food product comprising an antibiotic growth promoter (AGP) and lacking a FHP and MOS.

22. The method of claim 13, wherein the poultry are egg laying hens; and
whereby supplying to the egg laying hens the animal food product results in similar eggshell color values, a similar egg yolk, a similar percentage of shell, yolk, and egg white, or any combination thereof, of eggs laid by the egg laying hens as compared to eggs laid by comparable egg laying hens fed with a food product comprising an antibiotic growth promoter (AGP) and lacking a FHP and MOS.
